(19) United States
(12) Patent Application Publication
(10) Pub. No.: US 2025/0352091 A1
(43) Pub. Date: Nov. 20, 2025

(54) MEASUREMENT DEVICE FOR USE WITH AN INDWELLING CATHETER

(71) Applicant: Becton, Dickinson and Company, Franklin Lakes, NJ (US)

(72) Inventors: Adam J. Boud, Bluffdale, UT (US); Christopher Quach, North Salt Lake, UT (US); Haley Abramson, Costa Mesa, CA (US); Daniel Blanchard, Bountiful, UT (US); Chad Alan Tagge, Sandy, UT (US)

(21) Appl. No.: 19/207,101

(22) Filed: May 13, 2025

Related U.S. Application Data
(60) Provisional application No. 63/648,822, filed on May 17, 2024.

(51) Int. Cl. A61B 5/145; A61B 5/00

(52) U.S. Cl. CPC A61B 5/14503; A61B 5/6852; A61B 5/742; A61B 2560/0214; A61B 2560/0462; A61B 2562/168; A61B 2562/18; A61B 2562/227

(57) ABSTRACT

A measurement device may be used with an indwelling catheter. The measurement device can include a housing from which a stylet extends. The housing can include circuitry for receiving measurements from one or more sensors on the stylet. The circuitry may also include a wireless module for wirelessly transmitting the measurements to a patient's electronic medical record or monitoring device.

### RELATED APPLICATIONS

This application claims the benefit of U.S. Provisional Patent Application No. 63/648,822, which was filed on May 17, 2024, which is incorporated herein in its entirety.

### BACKGROUND

An arterial catheter may be indwelling to provide ongoing access to a patient's blood for withdrawal, infusion, measurement, etc. To perform measurements, a device such as a stylet may be inserted into the indwelling catheter.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a measurement device for use with an indwelling catheter. The measurement device can include a housing from which a stylet extends. The housing can include circuitry for receiving measurements from one or more sensors on the stylet. The circuitry may also include a wireless module for wirelessly transmitting the measurements to a patient's electronic medical record or monitoring device.

In some embodiments, a measurement device for use with an indwelling catheter may include a housing containing circuitry, a stylet that extends from the housing, and one or more sensors on the stylet. The circuitry may be configured to receive measurements from the one or more sensors.

In some embodiments, the circuitry may include a wireless module for wirelessly transmitting the measurements to an external system.

In some embodiments, the stylet may comprise a hollow interior through which one or more cables extend to connect the one or more sensors to the circuitry.

In some embodiments, the hollow interior may form a fluid pathway.

In some embodiments, the stylet may extend from the housing via a connector that is configured to connect the housing to a catheter adapter.

In some embodiments, the connector may be a luer connector.

In some embodiments, the circuitry may include a replaceable battery.

In some embodiments, the measurement device may include a medium that isolates the one or more sensors from an environment external to the stylet.

In some embodiments, the medium may be contained within one or more holes through a sidewall of the stylet and may be configured to allow an analyte to pass through to the one or more sensors.

In some embodiments, the medium may be positioned overtop the one or more sensors. In some embodiments, the housing may be wearable.

In some embodiments, the circuitry may include storage.

In some embodiments, the housing may include a display.

In some embodiments, the circuitry may include a wireless module.

In some embodiments, the circuitry may form a wired interface.

In some embodiments, the stylet may be configured to selectively occlude the indwelling catheter.

In some embodiments, a measurement device for use with an indwelling catheter may include a housing containing a wireless module, a stylet that is coupled to the housing, and one or more sensors positioned on the stylet. The one or more sensors may be configured to provide measurements to the wireless module for transmission to an external system.

In some embodiments, the housing may include a connector by which the housing is coupled to a catheter adapter.

In some embodiments, a measurement device for use with an indwelling catheter may include a stylet that is configured to insert into a catheter and to selectively occlude the catheter, one or more sensors on the stylet, and a wireless module that receives measurements from the one or more sensors and transmits the measurements to an external system.

In some embodiments, the stylet may have a hollow interior through which one or more cables extend to connect the one or more sensors to the wireless module.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIGS. 1A and 1B are side and cross-sectional views respectively of an example measurement device that is configured in accordance with one or more embodiments;

FIGS. 2A and 2B are side and cross-sectional views respectively of another example measurement device that is configured in accordance with one or more embodiments;

FIGS. 3A and 3B are perspective and cross-sectional views respectively of another example measurement device that is configured in accordance with one or more embodiments;

FIG. 4 provides another example of a measurement device that is configured in accordance with one or more embodiments including how the measure device may facilitate patient mobility; and

FIG. 5 provides an example of how a stylet of a measurement device may be configured to selectively occlude a catheter in accordance with one or more embodiments.

### DESCRIPTION OF EMBODIMENTS

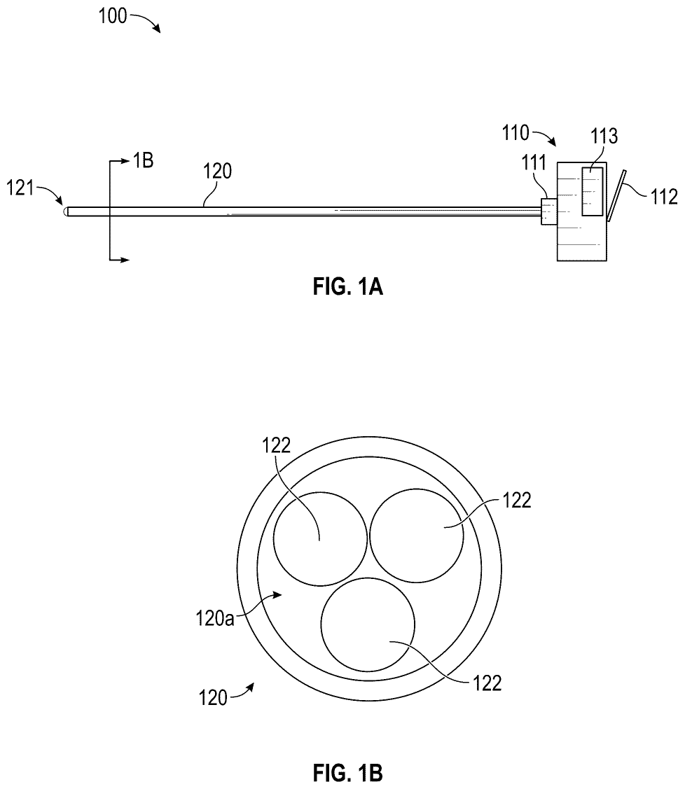

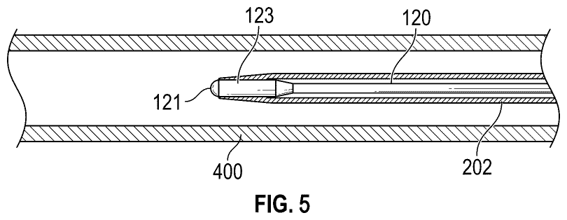

FIGS. 1A and 1B illustrate an example of a measurement device 100 that is configured in accordance with one or more embodiments. Measurement device 100 includes housing 110 and a stylet 120 that extends distally from housing 110 and that may include one or more sensors 121. Stylet 120 can be configured to selectively occlude a catheter into which it is inserted. For example, FIG. 5 provides an example where stylet 120 includes an occluding portion 123 that occludes a distal tip of a catheter 202 that is inserted into a patient's vasculature 400.

In some embodiments, stylet 120 may couple to housing 110 via a connector 111 (e.g., a luer connector) which can facilitate connecting housing 110 to a catheter into which stylet 120 is inserted (e.g., via a needle free connector on the catheter adapter). Stylet 120 could be formed of any suitable material such as polyimide, polyurethane, stainless steel, Nitinol, PEEK, etc.

In some embodiments, housing 110 may include a removable cover 112 which provides access to circuitry 113. In some embodiments, circuitry 113 can include one or more sensor base units (which may be in communication with one or more sensors 121 formed on or integrated into stylet 120), one or more wireless modules (e.g., for transmitting measurements), one or more batteries (e.g., for powering other components of circuitry 113), etc. Circuitry 113 can be configured to receive measurements obtained via one or more sensors on stylet 120 and to transmit these measurements to another system (e.g., to the patient's electronic medical record).

FIG. 1B, which is a cross-sectional view taken through stylet 120, provides an example of how stylet 120 may include a hollow interior 120a through which one or more cables 122 may be routed. Cable(s) 122 could be used for communicating with sensor(s) 121 included on stylet 120 at or towards the distal end of stylet 120 (e.g., as shown in FIG. 5). Any suitable number and/or type of sensor 121 could be used such as one or more pressure sensors, one or more temperature sensors, one or more optical sensors, one or more electromagnetic sensors, one or more electrochemical sensors, one or more photoelectric sensors, one or more electrodes, etc. In some embodiments, one or more of cable(s) 122 could be used to power corresponding sensor(s) 121. Various examples of sensors, sensor arrangements and stylet configurations for selectively occluding a catheter are provided in U.S. Provisional Patent Application No. 63/648,807, which is incorporated by reference.

In embodiments where circuitry 113 includes one or more wireless modules, measurement device 100 can be used to obtain measurements while allowing the patient to be mobile. For example, a patient may walk around while measurement device 100 remains connected to a catheter assembly and provides continuous monitoring. In such cases, the wireless module(s) can receive measurements from one or more sensors included on stylet 120 and can transmit the measurements to an external system (e.g., a nursing station). Any suitable wireless protocol could be used such as Wi-Fi, Bluetooth, NFC, etc.

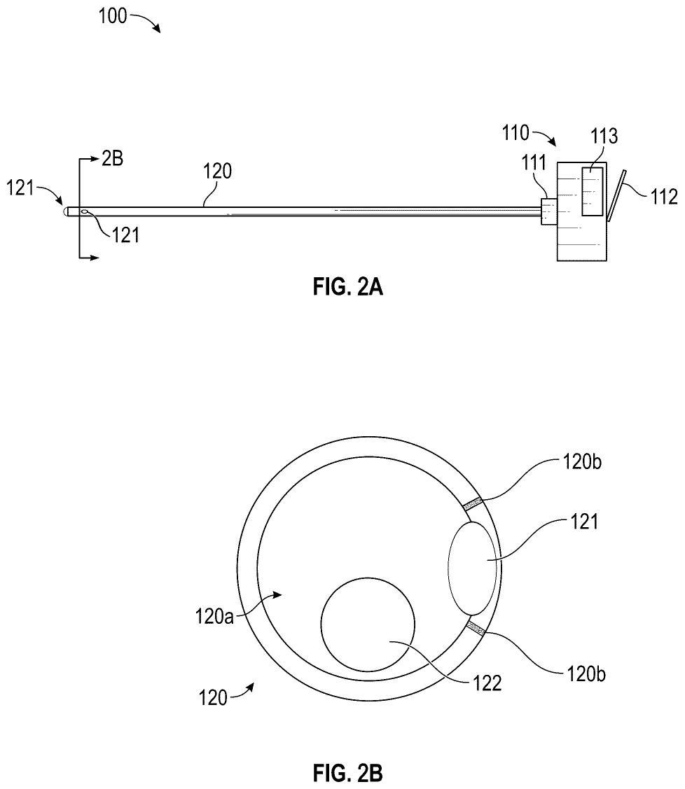

FIGS. 2A and 2B, which are similar to FIGS. 1A and 1B, provide an example of how stylet 120 may isolate one or more sensors 121. In this example, a sensor 121 is integrated into a sidewall of stylet 121 such that sensor 121 is not exposed to the external environment outside of stylet 121 (e.g., to blood, medicaments, other fluids, etc. that may be present in the patient's vasculature and/or within the catheter). To expose sensor 121 to a desired analyte, one or more holes 120b may be formed in stylet 121 in the vicinity of sensor 121. In some embodiments, each hole 120b may be filled with a medium that is diffusive to the analyte of interest and that is biocompatible. For example, a polyacrylamide gel could be used to fill holes 120b. In some embodiments, a polyacrylamide gel (or other suitable medium) could include additives that filter undesired analytes such that only the desired analyte is able to pass through holes 120b to reach sensor 121. In some embodiments, rather than or in addition to using one or more holes 120b filled with a medium, sensor 121 could be integrated into stylet 120 so that it forms part of the sidewall of stylet 120 (i.e., so that it is otherwise exposed to the external environment) and the medium can be used to directly cover sensor 121.

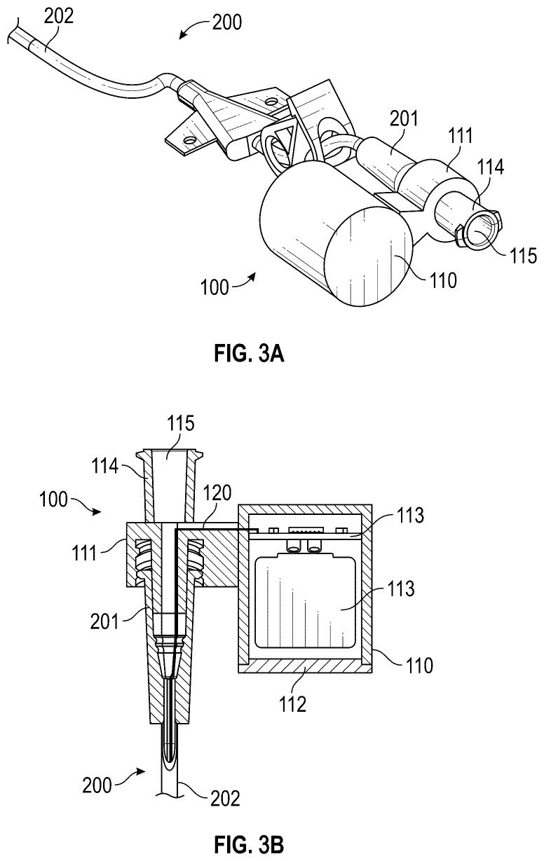

FIGS. 3A and 3B provide another example of how measurement device 100 may be configured in one or more embodiments. In this example, measurement device 100 includes a connector 111 that is connected to a connector 201 of a catheter assembly 200 from which a catheter 202 extends. Accordingly, stylet 120 extends through catheter 202 and may have its distal end positioned at or near a distal tip of catheter 202. In this example, an additional connector 114 is formed on (or extends from) connector 111 and provides an access point for fluidly connecting a blood draw device (or another device) to catheter 202. In particular, a fluid pathway 115 may be formed through connectors 111 and 114 and into the lumen of catheter 202.

As shown in FIG. 3B, stylet 120 may be routed from housing 110, through connector 111, and into catheter 202. FIG. 3B also provides an example of circuitry 113 that includes a circuit board to which the proximal end of stylet 120 (or, more specifically, the proximal end of cable(s) 122) are connected and a battery for providing power to the circuit board. The circuit board also includes various electronic components such as a wireless transceiver and one or more base units for sensors 121.

In some embodiments, stylet 120 could be configured to form a fluid pathway for performing blood draws and/or infusions. For example, in comparison to what is shown in FIG. 3B, the proximal end of stylet 120 could be connected to connector 114 so that fluid pathway 115 continues into hollow interior 120a of stylet 120 rather than into the lumen of catheter 202. In such cases, cable(s) 122 could be configured to extend through a sidewall of stylet 120 to connect to circuitry 113. The application incorporated by reference above provides examples of how the distal end of stylet 120 could be configured in these embodiments to enable blood draws or infusions through stylet 120. However, any suitable configuration of stylet 120 could be employed.

In some embodiments in which a fluid pathway is formed through stylet 120, housing 110 (including connector 111) could be configured to provide a separate fluid pathway into the lumen of catheter 202. For example, a separate connector could be provided to form this separate fluid pathway.

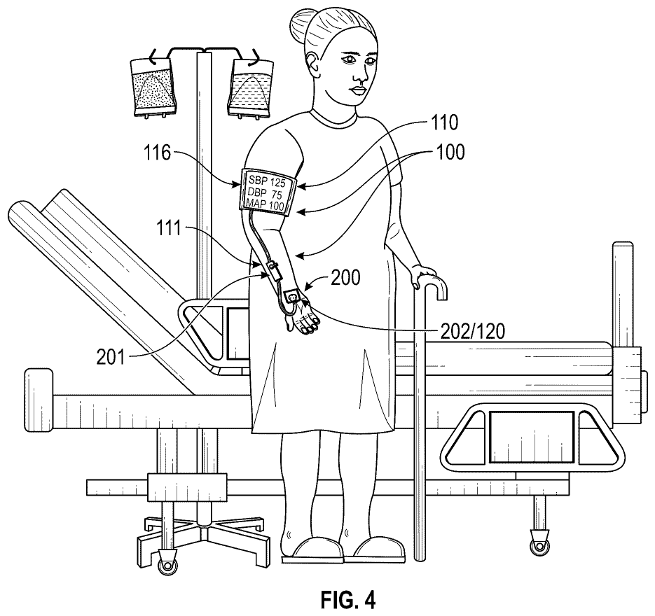

FIG. 4 provides an example where measurement device 100 includes a housing 110 that is in the form of an armband and is therefore wearable. Other types of wearable housings 110 could be employed such as a watch, adhesive patch, necklace, fanny pack, etc. In such embodiments, connector 111 could be connected to housing 110 via tubing (e.g., to house cables 122 and/or to form a fluid pathway) to facilitate a desired positioning of housing 110.

In some embodiments where housing 110 is wearable, housing 110 may include a display 116 on which measurements obtained via stylet 120 may be displayed in real-time. In some embodiments, circuitry 113 could employ display 116 to output alerts (e.g., similar to a vital signs monitor). In some embodiments, circuitry 113 could calculate advanced parameters from measurements obtained via stylet 120 and could output such advanced parameters (e.g., via display 116 or by transmitting the advanced parameters to an external system).

In some embodiments, circuitry 113 in a wearable housing 110 could include a wireless module for transmitting measurements to another system. In other embodiments, circuitry 113 of a wearable housing 110 may not include a wireless module but may include a wired interface for connecting to a nearby system such as a vital signs monitor. In such embodiments, circuitry 113 may also include storage for temporarily storing measurements while the wired interface is disconnected and may be configured to transfer stored measurements once the wired interface is again connected. In this way, patient mobility is still facilitated even though a wired interface is employed to transfer measurements from measurement device 100. Notably, because stylet 120 can be used to selectively occlude the catheter, the catheter does not need to remain connected to a saline bag and pressure tubing, which further enhances patient mobility.

A measurement device configured in accordance with one or more embodiments can be used to enhance mobility of any patient having an indwelling catheter. This enhancement in mobility may be due to the use of a wireless module to eliminate the need for a wired connection and/or to the use of a stylet that selectively occludes the catheter to eliminate the need for a constant flow of saline through the catheter. This mobility is provided without foregoing the benefits of constant monitoring and data acquisition.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A measurement device for use with an indwelling catheter, the measurement device comprising:
a housing containing circuitry;
a stylet that extends from the housing; and
one or more sensors on the stylet, the circuitry being configured to receive measurements from the one or more sensors.

2. The measurement device of claim 1, wherein the circuitry includes a wireless module for wirelessly transmitting the measurements to an external system.

3. The measurement device of claim 1, wherein the stylet comprises a hollow interior through which one or more cables extend to connect the one or more sensors to the circuitry.

4. The measurement device of claim 3, wherein the hollow interior forms a fluid pathway.

5. The measurement device of claim 1, wherein the stylet extends from the housing via a connector that is configured to connect the housing to a catheter adapter.

6. The measurement device of claim 5, wherein the connector is a luer connector.

7. The measurement device of claim 1, wherein the circuitry includes a replaceable battery.

8. The measurement device of claim 1, further comprising:
a medium that isolates the one or more sensors from an environment external to the stylet.

9. The measurement device of claim 8, wherein the medium is contained within one or more holes through a sidewall of the stylet and is configured to allow an analyte to pass through to the one or more sensors.

10. The measurement device of claim 8, wherein the medium is positioned overtop the one or more sensors.

11. The measurement device of claim 1, wherein the housing is wearable.

12. The measurement device of claim 11, wherein the circuitry includes storage.

13. The measurement device of claim 11, wherein the housing includes a display.

14. The measurement device of claim 11, wherein the circuitry includes a wireless module.

15. The measurement device of claim 11, wherein the circuitry forms a wired interface.

16. The measurement device of claim 1, wherein the stylet is configured to selectively occlude the indwelling catheter.

17. A measurement device for use with an indwelling catheter, the measurement device comprising:
a housing containing a wireless module;
a stylet that is coupled to the housing; and
one or more sensors positioned on the stylet, the one or more sensors being configured to provide measurements to the wireless module for transmission to an external system.

18. The measurement device of claim 17, wherein the housing includes a connector by which the housing is coupled to a catheter adapter.

19. A measurement device for use with an indwelling catheter, the measurement device comprising:
a stylet that is configured to insert into a catheter and to selectively occlude the catheter;
one or more sensors on the stylet; and
a wireless module that receives measurements from the one or more sensors and transmits the measurements to an external system.

20. The measurement device of claim 19, wherein the stylet has a hollow interior through which one or more cables extend to connect the one or more sensors to the wireless module.
